(19) United States
(12) Patent Application Publication
(10) Pub. No.: US 2026/0024638 A1
(43) Pub. Date: Jan. 22, 2026

(54) AI NURSE PLATFORM: AN ARTIFICIAL INTELLIGENCE- GENERATIVE-AI & DEEP LEARNING LARGE LANGUAGE MODEL/S (LLM) DRIVEN VIRTUAL ASSISTANT/S FOR ENHANCED PATIENT PERSONALIZED CARE OUTCOMES & FACILITY EFFICIENCIES

(71) Applicant: Abijith Kariguddaiah, Dublin, CA (US)

(72) Inventor: Abijith Kariguddaiah, Dublin, CA (US)

(21) Appl. No.: 18/777,782

(22) Filed: Jul. 19, 2024

(51) Int. Cl. G16H 20/10; G06N 3/098; G16H 10/60; G16H 40/20

(52) U.S. Cl. CPC G16H 20/10; G06N 3/098; G16H 10/60; G16H 40/20

(57) ABSTRACT

The present invention discloses AI Nurse, an advanced virtual assistant platform for healthcare. Its preferred embodiment utilizes AI, machine learning, and Large Language Models (LLMs) to provide real-time health assessments and personalized recommendations. AI Nurse supports various devices including wearables and integrates with AR/VR technologies and health platforms via APIs. It enhances patient outcomes by offering personalized Q&A assessments and event-specific recommendations, saving staff time and improving data control and analysis through blockchain integration. The platform facilitates near real-time care, early diagnosis, and patient empowerment over health data. Additional features include population health management, AR/VR integration, voice biometrics, telemedicine support, and multilingual capabilities. AI Nurse addresses healthcare challenges such as rural settings, aiming to streamline assessments and improve efficiencies for healthcare providers, ultimately enhancing overall patient care and outcomes.

### CROSS-REFERENCE TO RELATED APPLICATION

This non provisional application claims priority from US Continuation in Part patent application Ser. No. 17/474,090 filed on Sep. 14, 2021, and hereby claims the benefit of the embodiments therein and of the filing date thereof.

### FIELD OF THE INVENTION

The present invention relates to the field of natural language processing (NLP) and artificial intelligence (AI) in healthcare technology, and more specifically, to an AI/ML/NLP/GENAI/LLM-driven virtual assistant platform called AI Nurse that improves patient outcomes by providing automated Q&A assessments and monitoring vital health data, and combining with patients historical EMR (Electronic Medical Record) data, provides for near real time analysis and recommendations for the specific patient/event use case, providing new efficiencies to all constituents including the patient and clinic/hospital/Pharma/Insurance/health-organization clinicians. The platform utilizes artificial intelligence (AI) and machine learning (ML) & Generative-AI (GEN-AI) algorithms with deep learning (DL) Large Language models (LLM), which constantly learns to deliver personalized and real-time solutions to all constituents, especially the patients, and in multiple language and in their medium of choice (SMS/Voice/WhatsApp/etc.).

### BACKGROUND OF THE INVENTION

In the field of healthcare, patient outcomes are heavily influenced by the quality and timeliness of patient-nurse assessments and care interventions, for example in ambulatory post-surgical situations. A case can be made for many similar situations, e.g. in clinical trials, timely interventions become necessary while patients are trying out new unproven drugs, and especially for patients in home situations; Similarly, for most of rural American health settings, where care services can be poor and even emergency care is unavailable for hard-to-reach situations. Hence, due to various factors such as limited healthcare resources, nurse & staff shortages, time constraints, insurance coverage constraints, and constantly increasing need for patient health interventions, patients often face challenges in accessing timely and personalized care. i.e., initial nurse assessments just to investigate the adverse event and may have to wait days to weeks/months and emergency care is cost prohibitive to most, potentially worsening their health issues. And subsequent delays in discovery of the issues and successful interventions when it does arrive, leads to more complications and costs to the patient and entire health system. Clearly there is no near real time timely analysis of the patient's issue (collected data) for the same reasons and there is definitely no conjunction/investigation with the patient's historical data, which is never usually done due to the complexity and lack of bandwidth of the physicians to be able to access the required information with relevance and just in time when needed. Additionally, administrative tasks and the management of patient data, especially the accuracy of the data due to inherent human mistakes, can be cumbersome and time-consuming for healthcare providers, not to mention the costs. And in some instances, devastating to patient outcomes. In summary, lack of timely care most definitely leads to poorer outcomes in most cases, with today's solutions.

Existing solutions in the market offer limited capabilities in terms of AI-NLP based patient assessments & data. Let alone combining it with historical data, to provide rendered analysis & situational recommendations. There is a need for labour & cost efficiencies for the care providers and to alleviate & assist care provider staff from mundane tasks, convenience in patient engagement via multiple language and medium (Voice/SMS/WhatsApp) of choice, and AI analysis with integration with wearable devices for real time monitoring & care services. There is a need for an innovative platform that can leverage advanced AI/ML/NLP/GENAI/LLM algorithms, provide personalized assessments, personalized patient recommendations using deep learning LLMs, streamline administrative tasks, alleviate nurse shortage issues, and seamlessly integrate with various healthcare systems and wearable & non wearable devices for faster and in near real-time diagnosis and interventions with physician approvals, from a holistic perspective for superior patient outcomes.

### SUMMARY OF THE INVENTION

The proposed platform invention is an AI/ML/NLP/GENAI/LLM-driven virtual assistant platform called AI Nurse that addresses the aforementioned challenges and aims to improve patient outcomes. The platform provides custom automated Q&A assessments, patient-nurse engagement, situational analysis & recommendation and monitoring of vital health data. It is device-agnostic and can integrate with various wearable devices and technology & health platforms through APIs.

Key features of the platform include patient-nurse engagement, administrative time savings, data analysis & control, custom AI/ML/NLP/GENAI/LLM algorithms, cloud-based infrastructure, clinic/hospital/Pharma/Insurance/health-organization specific use cases, personalized emergency help, modular and plug-and-play integration, wearable & non wearable integrations, and blockchain integration and AI-driven assessments, analysis and recommendations.

The AI Nurse platform will support both Public and private LLMs where the public LLM is SaaS based and provides holistic view of all information regarding said area such as Gastroenterology, or Anesthesia or Orthopedic Surgery, or vascular surgery, etc., to name a few. Private LLMs will be provided for the larger establishments to have a closed loop secure data/analysis and historic views of the patients exclusively, and of the institution which manages it.

In addition to the key features, the platform offers additional features such as patient data analysis & control, AR/VR (Augmented Reality/Virtual reality) device integration and data analysis, adverse event monitoring, marketplace for assessments, AI/ML/NLP/GENAI/LLM-powered recommendations, monitoring and early diagnosis, and integration with AI platforms including Meta's Llama, Google's Gemini, Open Ais GPT-4 & Amazons Lex, and any other open AI platforms.

These features work together to provide a comprehensive solution that improves patient outcomes, enhances efficiency, and facilitates personalized care in healthcare settings. The platform leverages the power of AI/ML/NLP/GENAI/LLM and integrations with wearable & non wearable technology to enable collection of situational data, preventive care, early diagnosis, and just-in-time care interventions.

### BRIEF DESCRIPTION OF THE DRAWINGS

FIG. 1 is a schematic representation of the AI Nurse Assessment, analysis & recommendation platform architecture with the various modular components.

FIG. 2 is a flowchart illustrating the process of patient-nurse engagement and assessment generation using AI-NLP (Natural Language Processing).

FIG. 3 is a flowchart depicting the deep Learning LLM (Large Language Model) business process of both public and private LLMs for surgeries in this instant example.

FIG. 4 is a flowchart illustrating the further inner workings of a typical LLM (Large Language Model) and analysis process.

FIG. 5 is a flowchart demonstrating the Generative AI-driven rich content process flow.

FIG. 6 is a flowchart illustrating the wearables & non wearables integration and threshold analysis process.

FIG. 7 is a descriptive flowchart where the platform will integrate into the blockchain of choice to be able to secure all the static & dynamic transaction data.

FIG. 8 is a descriptive flowchart with the LLM “Heal GPT” Algorithmic flow of data collection and threshold pointer to access & train the AI.

### DETAILED DESCRIPTION OF THE INVENTION

The following detailed description of the invention provides a more thorough understanding of the various features and functionalities of the AI Nurse Assessment, analysis & recommendation platform. It should be noted that the invention is not limited to the specific embodiments described herein but can be implemented in various other forms for various types of healthcare organizational patient engagement use cases without departing from the scope of the invention.

### Architecture

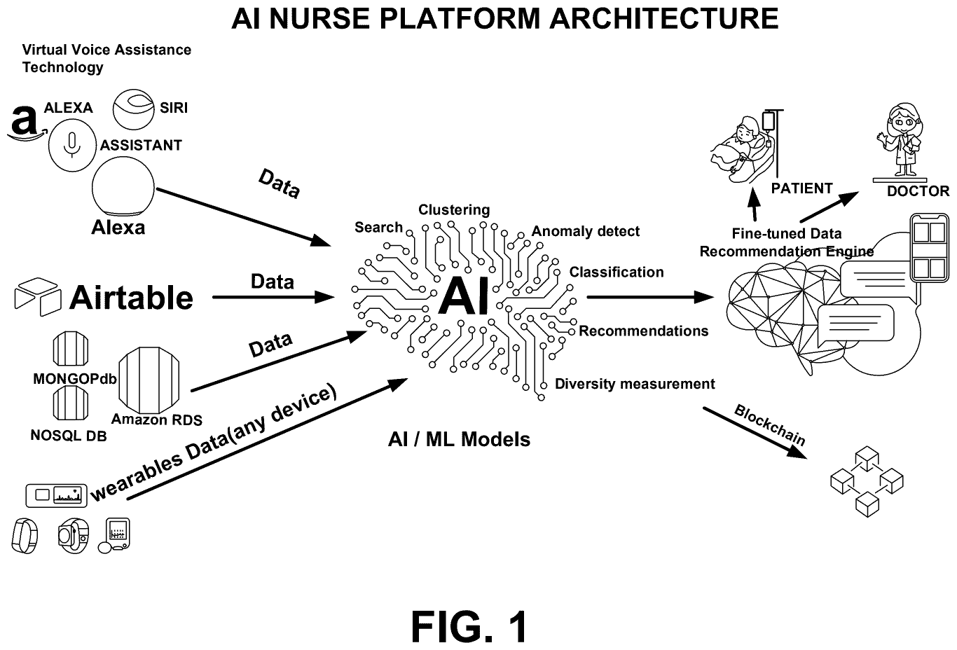

FIG. 1 illustrates the architecture of the AI Nurse Assessment, analysis & recommendation platform. The platform operates on a cloud-based infrastructure, which enables scalability, real-time data processing, and accessibility from multiple platform systems, and devices. The core component of the platform is an AI/ML/NLP/GENAI/LLM based algorithm/s that continuously learns and improves based on patient data and feedback, eliminating the need for direct contact between patients and nurses/staff for inquiries. And applicable to many use cases where the need for near real-time patient-engagement becomes a necessity for better outcomes

The platform integrates with various wearable & non wearable devices and health & other technology platforms through APIs, allowing seamless data exchange for monitoring, intelligent collating, analyzing and rendering recommendations for specific patient events to ensure best possible optimal health service & outcomes. Additionally, it utilizes blockchain technology to provide patients with control over their personal and health data.

As per one embodiment, the platform can utilize AI/ML/LLM based predictive analytics algorithms to forecast potential health risks and outcomes for individual patients. By analyzing historical patient data, demographic information, and other relevant factors, the platform can provide near real time insights and recommendations to healthcare providers for proactive interventions and personalized care plans. This applies to any healthcare areas that choose to utilize—examples include Gastroenterology, Orthopedics, Cardio, anesthesia, clinical trials of new drugs or therapies, vascular surgery, mental health, chronic pain management, etc.

### Patient-Nurse Engagement and Assessment Generation

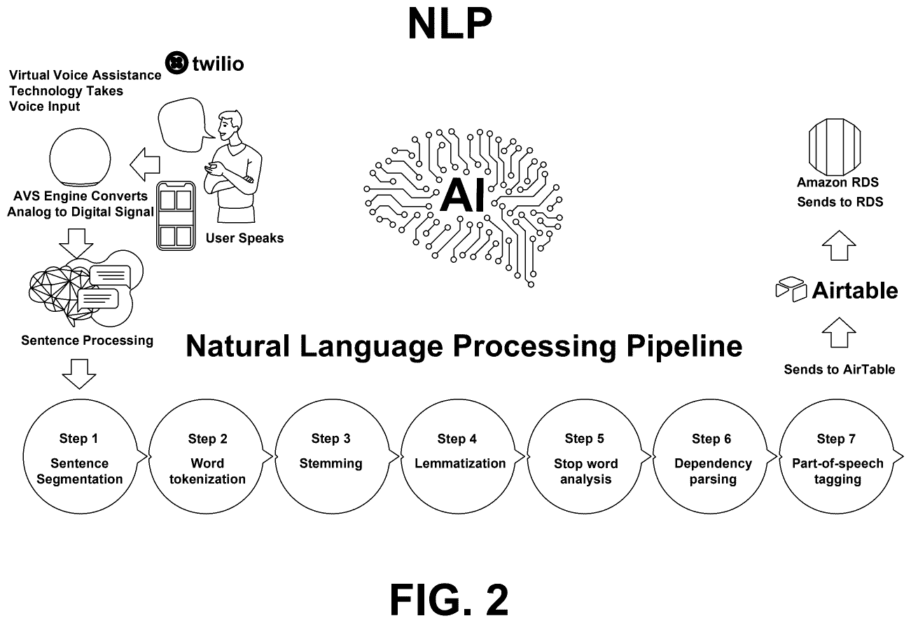

FIG. 2 presents a flowchart depicting the process of patient-nurse engagement and assessment generation. Patients can engage with nurses through choice of voice supported platforms, wearable & non wearable devices for various clinic/hospital/Pharma/Insurance/health-organization related interactions, including scheduling, pre/post procedure compliance, emergency, patient self-help and vitals checks & analysis (i.e. an instant on-demand health check analysis, where patient gets a status on their current health based on all available data for personalized health)

The platform utilizes natural language processing (NLP) techniques to interact & understand patient queries, generate custom appropriate assessments (e.g. based on underlying conditions), captures and provide responses to care team, and clinic approved recommendations to patients. Platform utilizes Custom AI/ML/NLP/GENAI/LLM algorithms from providers like OpenAI (GPT-4), Amazon Lex, Microsoft, IBM, and Google AI and incorporated to enhance patient outcomes and automate Q&A assessments, analysis & recommendations.

As per an embodiment, the platform can integrate seamlessly with EHR (Electronic Health Record) systems to access and update patient data in real-time, per the use case requirements. This integration allows the platform to enable healthcare providers and organizations to have a holistic view of the patient's medical history, current assessments, analysis and recommended actions, enhancing continuity of care and reducing the risk of errors or omissions.

Another embodiment, in addition to understanding patient queries and generating responses, the platform can incorporate NLG techniques to generate patient-friendly reports and summaries. NLG algorithms can convert complex medical information into easily understandable narratives, improving patient engagement and comprehension of their health conditions and recommended actions.

### Nurse & Administrative Time Savings

The platform offers assessments for clinic/hospital/Pharma/Insurance/health-organization compliance, specific custom use cases, such as scheduling patient appointments, custom patient queries, complex assessments, and conducting patient reviews on specific aspects (procedure, staff, etc.). These processes are automated, saving nurse & administrative time and increasing efficiency. The platform supports voice commands, calendar integration, and SMS for automated scheduling, review and clinic/hospital/Pharma/Insurance/health-organization specific use cases, which also includes underlying conditions such as diabetes or Heart conditions amongst others.

As per an embodiment, the platform can integrate with medication management systems and utilize AI algorithms to monitor patient medication adherence, and/or any side effects as part of most surgical or medical procedures. By analyzing data from pharmacies, pill dispensers, smart medication packaging, or connected medication apps, the platform can identify non-adherence patterns and send reminders or notifications to patients to improve medication compliance and other interventions catering to the care teams for corrective actions

As per an embodiment, the platform can support clinical trials for new drugs or therapies where patients are engaged via voice interactions with continuous assessments to ensure the trail parameters are adhered to including medication adherence, and also allow for routine or emergency situations to be handled in near real time, reducing patient complications and in the most efficacious manner that lowers costs and improves the clinical trial outcomes. Furthermore, the private deep learning LLM for said clinical trial allows for data analysis and sharing and ability to answer all questions to make for a more expedient clinical trial with audit trail & documentations to support the trail, while meeting compliance requirements.

As per an embodiment, the AI platform will offer all surgery and procedures that require anesthesia with perioperative/pre anesthesia checks and assessments (and post procedure long-term checks) per compliance requirements, and along with patients EHR data and other available data, offer the risk assessment for patient for the anesthesia per ASA (American Society of Anesthesiology) perioperative risk classification and other required surgery risks assessments as necessitated by the specific surgery (examples include Frailty Scores, RCRI-revised cardiac risk index, Sleep Apnea Assessment, Cognitive Dysfunction, COPD, Ain Management Risk, etc.), thereby assisting anesthesiologist to be able to make their decisions more accurately and in a timely fashion, and vastly improving patient outcomes and nurse & anesthesia staff efficiencies, and take on more case loads

As per an embodiment, the platform will offer all surgery and procedures that require pain management (including chronic) and medication side effects checks and assessments and along with patients EHR data and other available data, offer the risk assessment for patient for the said medications and constant checks, per compliance, thereby assisting pain specialists to be able to make any changes to their decisions faster, and cater to any changes in the medications etc.

### Data Control and Blockchain Integration

Patients have control over their personal and health data through blockchain technology. The platform enables the storage, access, and availability of patient data as Non-Fungible Tokens (NFTs) along with membership TOKENS tied to specific categories of health data. This empowers patients to control their data and access said data and monetization, ensuring privacy and security.

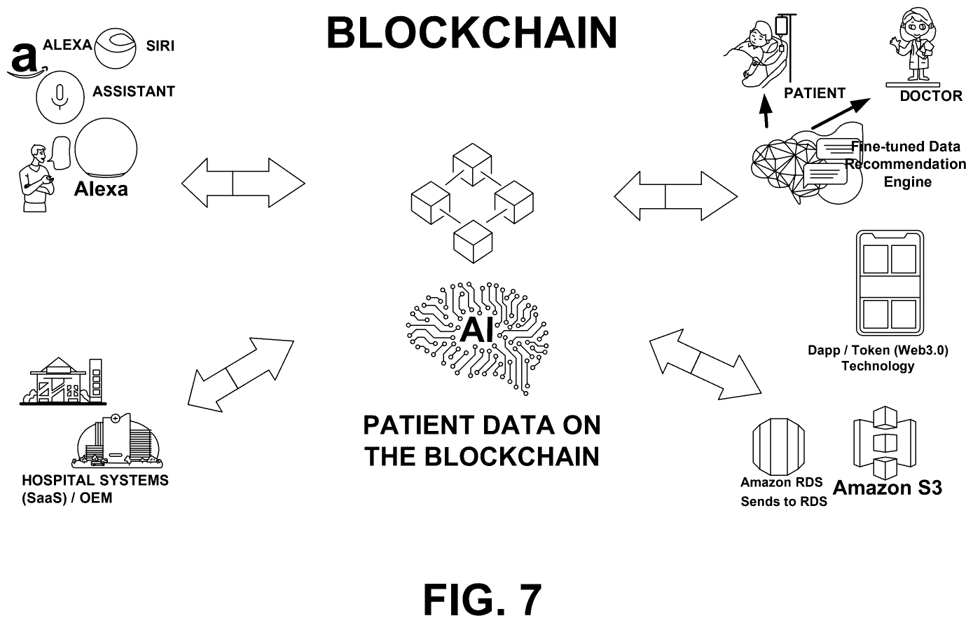

FIG. 7 illustrates the data control and blockchain integration process. Patient data is stored on the blockchain, and patients can determine who has access to their data and who can view or purchase specific information. The integration of blockchain technology provides transparency, immutability, and data ownership to patients.

### LLMs—Large Language Models

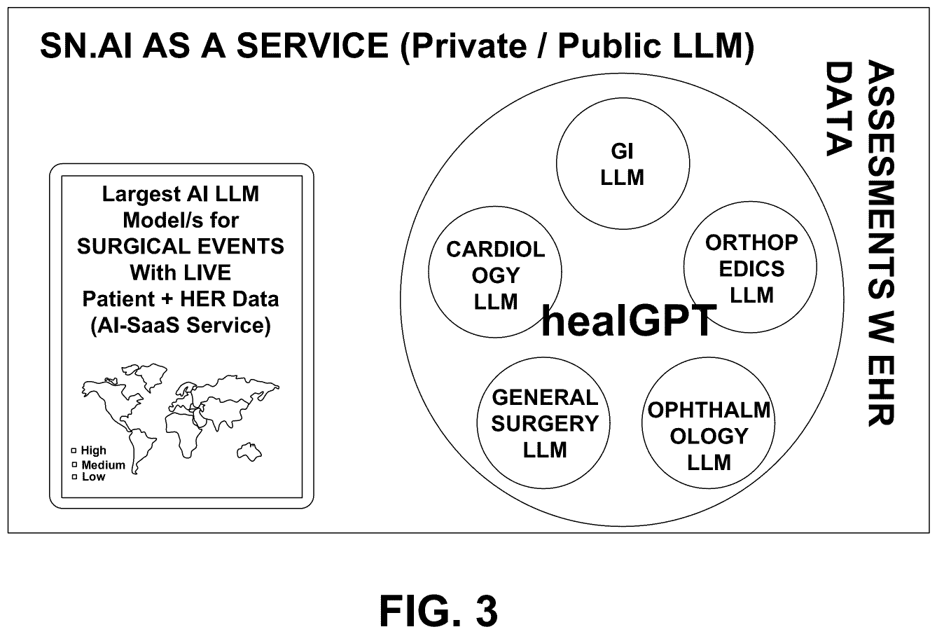

FIG. 3 depicts AI Nurse platform's deep learnings LLM offerings which include private and Public LLMs. The deep learning LLMs cater to their constituents by constantly learning and being able to answer more and more sophisticated queries and is directly proportional to time and quality data harnessing & consumption. The main difference being 1. Private llms are catering to the health organization and its patients and internal workforce, While the public LLM s a generic representation of the models inherent to the specific use case (e.g. Wound care in Gastroenterology, or wound care in orthopedic surgeries, etc.) and caters to the consuming public via a public SaaS transaction-based approach.

### Wearable & Non-Wearable RPM Integration and Analysis

The platform supports the integration of Remote Patient Monitoring (RPM) solutions, allowing patients and clinic/hospital/Pharma/Insurance/health-organization to integrate various health vitals monitoring devices such as EKG monitors, glucose monitors, sleep monitors, etc. This integration enhances the range of health data available for analysis, making RPM device agnostic vis custom APIs and improves outcomes.

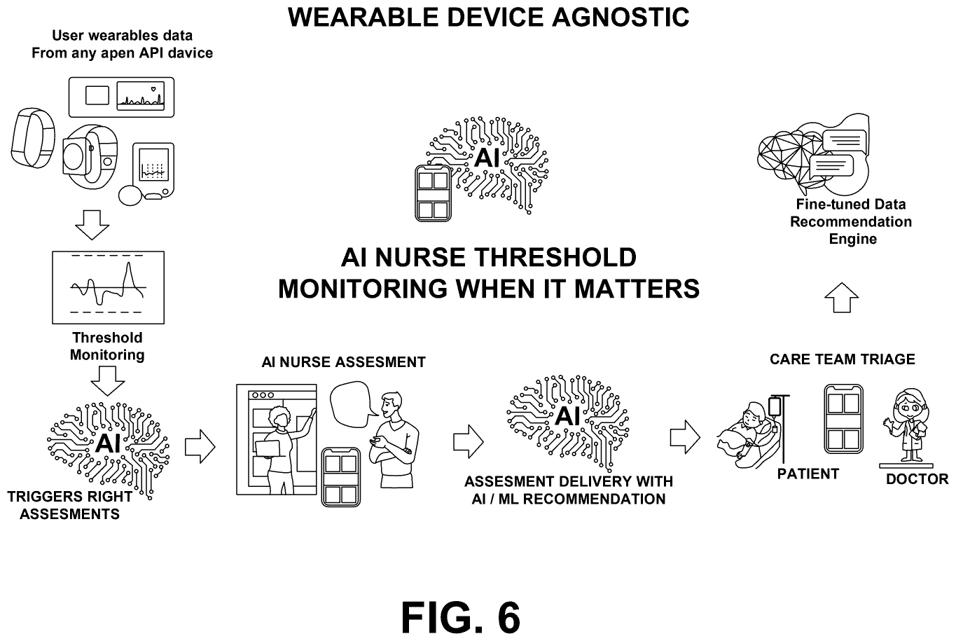

FIG. 6 presents a flowchart depicting the RPM integration and analysis process. RPM devices transmit health vitals data to the platform, which is then analyzed using AI/ML/NLP/GENAI/LLM algorithms. The platform generates insights, triggers assessments if necessary, and provides timely information to care teams for specific and immediate responses to adverse events.

### AI-Driven Assessments and Recommendations

The platform utilizes AI/ML/NLP/GENAI/LLM/NLP/GENAI/LLM models with multiple sources of data including assessment data, patient specific historic EMR data, clinic/hospital/Pharma/Insurance/health-organization data and proven and validated public information to assess and recommend actions based on real-time sensory data and pre-defined algorithms. The AI powered chatbots offers human-like conversations and the patient-nurse assessment Q&A fed into the AI engine for the specific adverse event/case/trauma being experienced by patient, and produce a recommendation for the doctor to review, and upon approval it is delivered to the patient as recommended solution for their adverse event. The AI recommended information will also provide for ‘Generative AI’ information, which include rich media (audio, video, PDFs, etc.) content, doctor's-indications, public-verified information, etc. relevant to the specific adverse event. The information and recommendations provide for just-in-time care, early diagnosis and better patient outcomes.

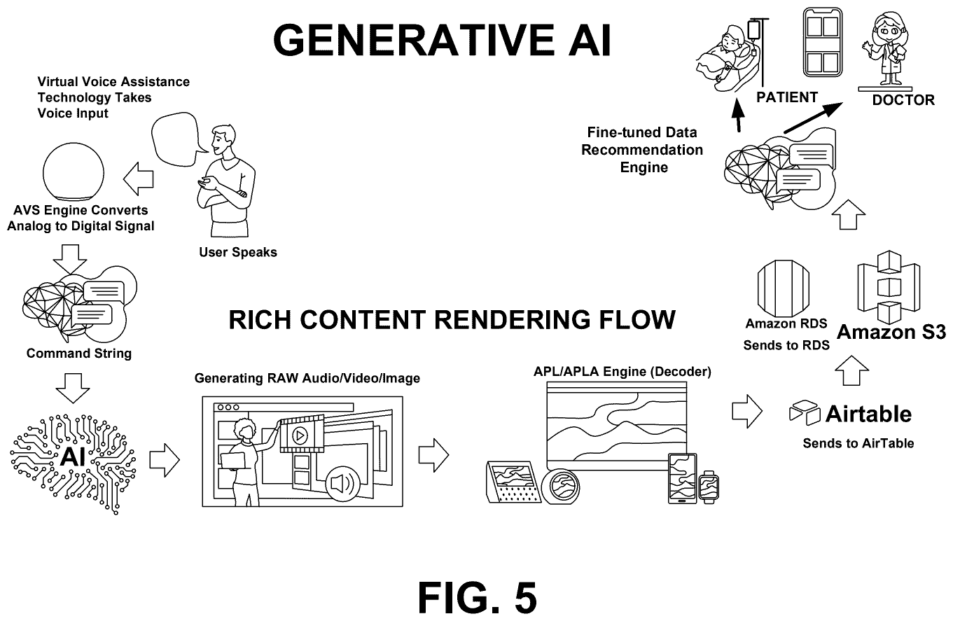

FIG. 5 illustrates the AI-driven assessment and recommendation process. Real-time sensory data from wearable devices is analyzed by the AI/ML/NLP/GENAI/LLM models, which generate assessments and recommendations based on patient-specific conditions. The platform ensures a human-like conversational AI experience during patient-nurse engagements through advanced NLP techniques.

### Monitoring and Early Diagnosis

By leveraging real-time sensory data from wearable devices and applying AI/ML/NLP/GENAI/LLM algorithms, the platform can determine the probability of infections and facilitate Q&A sessions and triage with healthcare personnel for early diagnosis and preventive care. This proactive approach improves patient outcomes and reduces the burden on healthcare resources.

### Smart Wearables and Non-Wearables

The platform integrates with various sensors, including EKG sensors, pulse detection sensors, and other health monitoring devices. It utilizes AI/ML/NLP/GENAI/LLM technologies to analyze user data and provide better outcomes for patients, insurers, and healthcare establishments. The platform's compatibility with both wearable and non-wearable devices ensures flexibility and accessibility for a wide range of patients.

### Marketplace for Assessments

The platform includes an independent marketplace where clinics, hospitals, Pharma, Insurance, health-organizations, and medical professionals can register and subscribe to AI Nurse platform services. This marketplace enables on-demand access to just-in-time care assessment & recommendation services, facilitating efficient near real time, and cost-effective healthcare delivery.

### Patient Data Control

The platform allows patients to have control over their personal health data by storing it on the blockchain as Non-Fungible Tokens (NFTs). Patients can determine via membership tokens who has access to their data and who can view or purchase specific information. This feature ensures privacy, transparency, and patient autonomy.

FIG. 7 illustrates the patient's ability to control their data on the blockchain, giving them ability to monetize their data for parties wanting such data for research purposes.

### Device Integration

The platform supports seamless integration with wearables and non-wearable remote patient monitoring (RPM) devices such as EKG monitors, health vitals trackers, blood glucose monitors, apple watch, AR/VR devices etc. This integration allows patients and clinic/hospital/Pharma/Insurance/health-organization to include patent health RPM data in their assessments, providing a comprehensive view of patient health and enabling more accurate recommendations.

### Modular and Plug-and-Play

The platform is designed to be modular and can be easily integrated into existing corporate health systems, mobile, web-based applications, wearable or non-wearable cloud-based (integrated) devices. The AI Nurse Assessment, analysis & recommendation module/s can be used as a plug-and-play solution, offering its features through an OEM licensing and/or software-as-a-service (SaaS) model. This modularity and compatibility enable seamless adoption and utilization of the platform across various healthcare settings.

### Routine & Adverse Event Monitoring

The platform monitors routine or adverse events in real-time and triggers assessments via physiological vitals threshold monitoring to provide care teams with timely information. This enables specific and immediate responses to routine checkups or adverse events, leading to better patient outcomes. By proactively monitoring and addressing routine checks or adverse events, the platform improves patient safety and reduces the risk of complications.

FIG. 6 is a flowchart illustrating the wearables & non wearables integration and threshold analysis process; When the patients vitals which can be continuously monitored crosses the thresholds that's been set/assigned, it triggers the routine or custom assessments and next steps to ensure care team is informed to take on the necessary steps to address the health event ensuring better outcomes.

### Data Collection

At the nucleus of this inventive paradigm, the AI Nurse application instigates the procedural cascade by engaging in the systematic compilation of precise use case assessment data. This encompassing data may include, but is not limited to, particulars regarding the specific emergency or medical issue confronting the patient, for instance, adverse effects stemming from medication consumption. The data collection process is designed to be perceptive and comprehensive, ensuring the capture of pertinent information essential for the ensuing analytical phases.

### Intelligent Data Aggregation

Subsequently, the AI engine assumes the role of data custodian, harmoniously amalgamating the recently accrued data with a trove of historical patient information. This amalgamation results in the formation of an extensive and holistic dataset, thereby affording a more profound and insightful basis for subsequent analytical proceedings. The objective herein is to form an inclusive repository that accounts for the historical context of the patient's medical journey, allowing for a multifaceted analysis.

### Recommendation Generation Using Deep Learning LLMs

Leveraging state-of-the-art artificial intelligence algorithms and methodologies, the AI engine undertakes the meticulous scrutiny of the amalgamated dataset. This rigorous analytical endeavor aims to unravel latent patterns, correlations, and anomalies within the dataset, culminating in the formulation of meticulously customized recommendations. These recommendations are constantly updated with new learnings, and judiciously tailored to the specific use case and are characterized by their adaptability to the dynamic nature of the patient's medical condition.

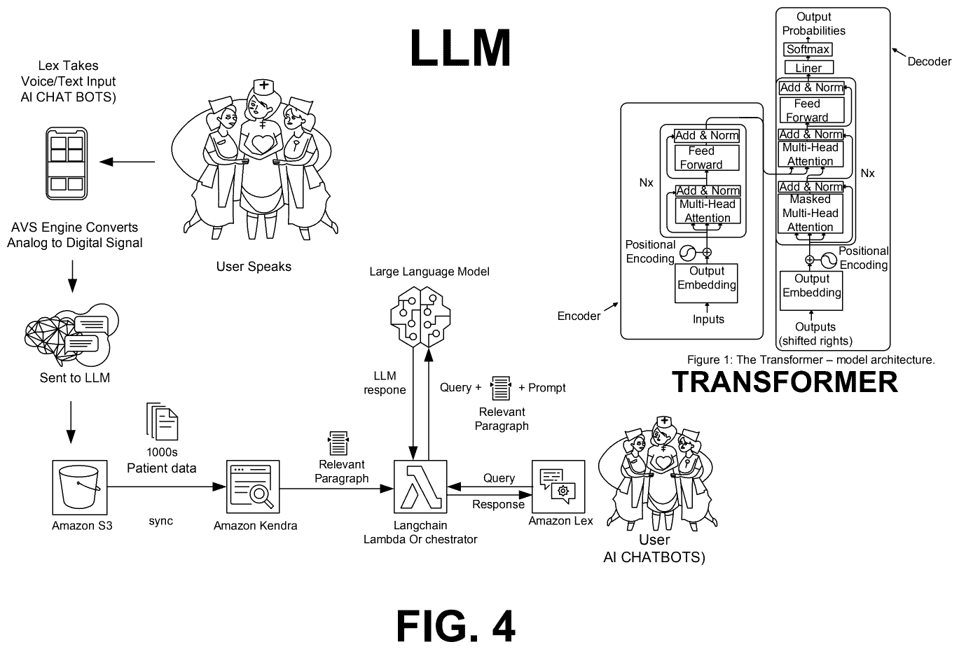

FIG. 4 illustrates the specialized LLM-Heal GPT's Algorithmic flow of data collection and threshold pointer to access & train the AI. The Heal GPT Algorithm for Personalized Healthcare Recommendations comprises a series of steps and components, shown

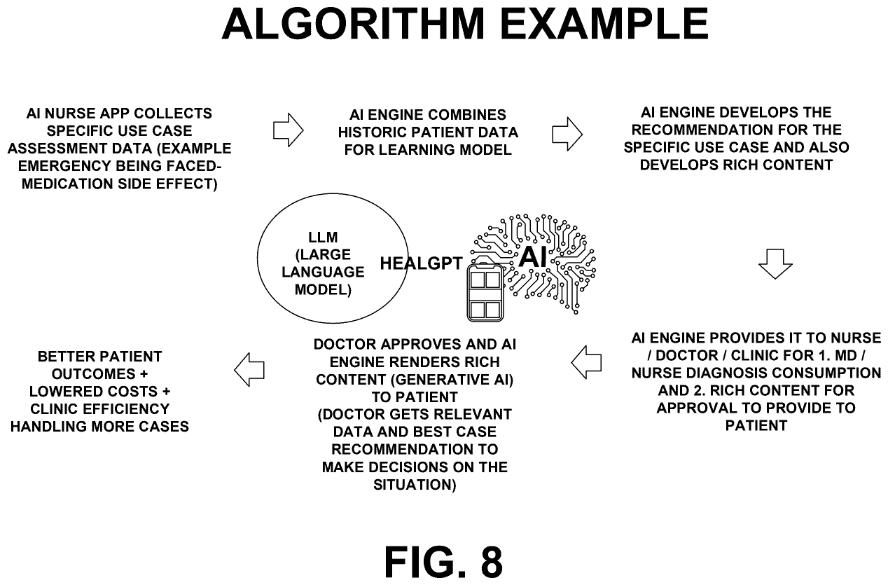

FIG. 8 illustrates an example use case for the specialized LLM-Heal GPT's Algorithmic flow of data collection and process flow in rendering the Healthcare Recommendations and the various steps comprising the process.

### Content Development

Concomitant to the recommendation generation process, the AI engine diligently orchestrates the creation of an expansive repository of rich content. This comprehensive content is replete with in-depth insights, elucidations, and educational materials that are intricately aligned with the patient's particular medical condition. It is designed to be informative, accessible, and conducive to enhancing the patient's understanding of their ailment and available treatment options.

FIG. 5 illustrates the rich content rendering workflows; the ability for the AI engine to render custom contextualized rich audio and video content to benefit the clinics and the patients, so that they are better educated, become easily compliant and have a superior patient satisfaction & experience.

### Medical Professional Review

Subsequent to the formulation of the AI-generated recommendations and the assembly of rich content, these assets are conscientiously channeled to the purview of esteemed medical professionals, including but not limited to physicians and nurses. The role of these healthcare experts is to engage in a thorough assessment and evaluation of the recommendations and accompanying content. Their expertise and discernment serve as a critical checkpoint in ensuring the accuracy, appropriateness, and relevance of the information and recommendations provided.

### Approval and Patient Delivery

Following a rigorous and informed review by medical professionals, an authorization process ensues. Herein, medical professionals possess the prerogative to endorse and approve the AI-generated recommendations and associated content for distribution to the patient. In instances where approval is granted, the AI engine orchestrates the seamless dissemination of the rich content to the intended patient. This meticulous process is executed with the utmost precision to guarantee that the patient is equipped with the necessary information to comprehensively grasp their medical condition and make informed decisions regarding their treatment path.

The below addressed are additional embodiments further enhance the capabilities and versatility of the AI Nurse Assessment, analysis & recommendation platform, enabling it to address a wider range of healthcare challenges and provide tailored solutions for improved patient outcomes and healthcare efficiency, such as:

The platform can be extended to support population health management initiatives by aggregating anonymized patient data from multiple sources. By applying AI/ML/NLP/GENAI/LLM algorithms to this data, the platform can identify trends, patterns, and risk factors at the population level, helping healthcare organizations make informed decisions regarding resource allocation, preventive measures, and targeted interventions.

The platform can be extended to rural area and underserved community scenarios and situations where the access to medical services and access to healthcare is not easily available or it can be extended to situation where it takes longer time to receive medical care and assistance due to distance and resource shortages like nurse, doctor or healthcare services. The ability for a remote patient to receive a virtual nurse assessment about the health issue being faced and an AI based interim temporary doctor approved intervention recommendation (in near real time), to avert disastrous events, would be invaluable for the entire care value chain, and ensures better patient outcomes.

The platform can incorporate AR/VR technology to provide immersive and interactive experiences for patients during assessments and consultations. AI nurse platform will facilitate AR/VR simulations to be used to educate patients about their health conditions, demonstrate medical procedures, or offer relaxation techniques for pain management and stress reduction.

The platform can utilize voice biometrics technology to authenticate patients during interactions and ensure data privacy and security. By analyzing unique vocal characteristics, the platform can verify the identity of the patient, protecting sensitive health information from unauthorized access.

The AI Nurse platform can integrate with telemedicine platforms to enable seamless virtual consultations between patients and healthcare providers. By facilitating video conferencing, real-time data sharing, and AI-driven assessments, the platform enhances the effectiveness and efficiency of remote healthcare delivery.

The AI/ML/NLP/GENAI/LLM algorithms powering the platform can be designed to continually learn from new data, new learnings and update their models to improve accuracy and relevance over time. The platform can leverage federated learning techniques to aggregate and analyze data from multiple healthcare providers while preserving data privacy and security.

The platform can be expanded to provide multilingual support, allowing patients to interact with the AI Nurse virtual assistant in their preferred language. This feature enhances accessibility and inclusivity, ensuring that language barriers do not hinder effective communication and understanding between patients and healthcare providers.

The AI Nurse Assessment, analysis & recommendation platform provides a comprehensive solution for improving patient outcomes through AI-driven assessments, personalized routine or emergency help, and modular integrations. By leveraging advanced AI/ML/NLP/GENAI/LLM algorithms, wearable & non-wearable technology, and blockchain integration, the platform aims to vastly enhance efficiency, convenience, and patient care in healthcare settings.

This inventive system, through its holistic integration of data collection, analysis, and recommendation generation, emerges as an invaluable tool in the healthcare landscape. By virtue of its capacity to deliver personalized care and tailor medical recommendations based on real-time assessments and historical patient data, the Heal GPT Algorithm for Personalized Healthcare Recommendations redefines healthcare practices, augments patient outcomes, ameliorates cost-efficiency, and enhances clinic/hospital/Pharma/Insurance/health-organization operations. Its ability to seamlessly adapt to the unique needs of each patient contributes to the realization of a more refined and patient-centric healthcare paradigm.

While the invention has been described with reference to specific embodiments, it should be understood that the embodiments are illustrative and not restrictive. An example-AI Nurse will support instant patient review instigated by either patient for self-care or by care team, as long as they and their data are on the platform, to be able to provide for a wholistic health check or specific area related health checks for instant peace-of-mind. Various modifications and changes may be made by those skilled in the art without departing from the scope of the invention. Therefore, the scope of the invention should be determined by the appended claims and their legal equivalents.

I claim:

1. An AI platform system for capturing patient data and providing personalized recommendations, comprising:
a. Initiating data collection by deploying an AI Nurse platform application to gather specific use case assessment data, wherein said data relates to any non-emergency (e.g. adverse event, routine checkups) as well as emergency situations encountered by a patient, including, but not limited to, instances involving medication side effects.
b. Intelligently integrating said collected data with historical patient information from external sources such as EHR & other health systems, thereby establishing a comprehensive dataset for subsequent analysis, said intelligent gathering performed by an AI platform engine.
c. Utilizing advanced artificial intelligence algorithms, performing data analysis using said AI platform engine to generate personalized recommendations tailored to the specific use case.
d. Concurrently generating and rendering relevant rich content, including detailed information and educational audio/video materials related to the patient's medical event/condition, employing the same AI platform engine.
e. Providing AI-generated recommendations and rich content to medical professionals, including but not limited to doctors, nurses, admin staff, for evaluation and assessment, and consumption.
f. Facilitating approval of the generated recommendations and content by said medical professionals for subsequent consumption by the patient & family; and
g. Delivering said approved rich content to the patient, thereby ensuring the provisioning of pertinent information pertaining to the patient's medical event/condition and available treatment options for better outcomes.

2. An AI/ML/NLP/GENAI/LLM-driven virtual assistant called AI Nurse, comprising:
a. A cloud-based infrastructure for scalability, real-time data processing, and accessibility
b. An AI platform for near real-time communication offered via voice, in multiple languages, and any other communication of choice including SMS, WhatsApp, skype, etc.
c. An AI/ML/NLP/GENAI/LLM based algorithm that continually learns and improves based on patient data and feedback.
d. Integration with wearable & non wearable devices and health and technology platforms through APIs for seamless data exchange and monitoring of vital health data.
e. Deployable on any compatible external edge platforms including existing mobile, web applications, wearable devices such as apple watch or A/VR devoices like Oculus e. Blockchain technology for providing patients control over their personal and health data.

3. The system of claim 2, further comprising:
a. Natural language processing (NLP) techniques for understanding patient queries, generating appropriate custom assessments, and providing analysis responses or recommendations.
b. Integration with electronic health record (EHR) systems for accessing and updating patient data in real-time.
c. NLG techniques for generating patient-friendly reports and summaries.

4. The system of claim 2, wherein the platform automates clinic/hospital/Pharma/Insurance/health-organization specific use cases, including scheduling patient appointments and conducting patient assessments/reviews, thereby saving nurses/administrative time and increasing efficiency.

5. The system of claim 2, wherein patients have control over their personal and health data through blockchain technology, enabling storage, access, and availability of patient data as Non-Fungible Tokens (NFTs) tied to specific categories of health data.

6. The system of claim 2, further comprising integration with wearable & non wearable Remote Patient Monitoring (RPM) solutions, allowing integration of various health vitals monitoring devices and analysis of the transmitted health vitals data using AI/ML/NLP/GENAI/LLM algorithms.

7. The system of claim 2, wherein AI/ML/NLP/GENAI/LLM models assess and recommend actions based on intelligently gathered real-time sensory, EHR data and pre-defined algorithms, generating personalized questions for patients, triggering Q&A sessions with patients and/or healthcare personnel, and providing analyzed answers and recommendations for just-in-time care and early diagnosis.

8. The system of claim 2, wherein real-time sensory data from wearable & non wearable devices is analyzed by AI/ML/NLP/GENAI/LLM models to determine appropriate care responses for any adverse events, including the probability of infections, facilitating Q&A sessions and triage with healthcare personnel for early diagnosis and preventive care.

9. The system of claim 2, further comprising seamless integration with sensors, wearable & non-wearable remote patient monitoring (RPM) devices & smart solutions such as smart rings, AR/VR, enabling inclusion and analysis of such patients health data in assessments for a comprehensive view of patient health and accurate recommendations, and providing better outcomes for patients, insurers, and healthcare establishments.

10. The system of claim 2, comprising an independent marketplace for clinics, hospitals, Pharmaceuticals, Insurance, health-organizations, and medical professionals to register and subscribe to AI Nurse custom assessment, analysis & recommendation services and facilitating on-demand access to just-in-time care assessment, analysis & recommendation services for any situation/s for faster on-demand care.

11. The system of claim 2, wherein the platform is modular and can be easily integrated into existing health systems, mobile or web-based applications, or wearables, offering its features through a software-as-a-service (SaaS) model.

12. The system of claim 2, wherein routine or adverse events are monitored in real-time, triggering assessments to provide care teams with timely information for specific and immediate responses, thereby improving patient outcomes and safety.

13. The system of claim 2, wherein the platform aggregates anonymized patient data from multiple sources using AI/ML/NLP/GENAI/LLM algorithms, identifying trends, patterns, and risk factors at the population level to support population health management initiatives.

14. The system of claim 2, wherein AI Nurse platform will support instant patient review instigated by either patient for self-care or by care team, as long as they and their data are on the platform, to be able to provide for a wholistic health check or specific area related health checks for instant peace-of-mind.

15. The system of claim 2, wherein the platform incorporates AR/VR technology to provide immersive and interactive experiences for patients during assessments and consultations, facilitating education, medical procedure demonstration, and relaxation techniques.

16. The system of claim 2, wherein voice biometrics technology is utilized to authenticate patients during interactions, ensuring data privacy and security by analyzing unique vocal characteristics.

17. The system of claim 2, further comprising seamless integration with telemedicine platforms for seamless virtual consultations between patients and healthcare providers, enhancing the effectiveness and efficiency of remote healthcare delivery.

18. The system of claim 2, wherein AI/ML/NLP/GENAI/LLM based algorithms are designed to continually learn from new data and update their models to improve accuracy and relevance, leveraging federated learning techniques for preserving data privacy and security.

19. The system of claim 2, providing multilingual support, allowing patients to interact via voice with the AI Nurse Assessment, analysis & recommendation platform's virtual assistant in their preferred language, enhancing accessibility and inclusivity.

20. The system of claim 2, providing multimodal integration support, allowing patients to interact via any device or location of choice, e.g. Alexa in the car or Alexa on the TV, or on wearable devices or standalone edge devices, with the AI Nurse Assessment, analysis & recommendation platform's virtual assistant in their preferred language, enhancing accessibility and inclusivity.
